(19) United States
(12) Patent Application Publication
(10) Pub. No.: US 2026/0272449 A1
(43) Pub. Date: Sep. 17, 2026

(54) SYSTEMS AND METHODS FOR LEAFLET FOLDING

(71) Applicant: Edwards Lifesciences Corporation, Irvine, CA (US)

(72) Inventors: Noa Axelrod Manela, Kadima (IL); Eitan Atias, Tel Aviv (IL); Ofir Witzman, Harish (IL)

(73) Assignee: Edwards Lifesciences Corporation, Irvine, CA (US)

(21) Appl. No.: 19/667,505

(22) Filed: May 4, 2026

Related U.S. Application Data
(63) Continuation of application No. PCT/US2024/055891, filed on Nov. 14, 2024.
(60) Provisional application No. 63/600,551, filed on Nov. 17, 2023.

(51) Int. Cl. A61B 17/04; A61B 17/00; A61B 17/34

(52) U.S. Cl. CPC A61B 17/0469; A61B 17/00234; A61B 17/34; A61B 2017/00243; A61B 2017/003; A61B 2017/00353; A61B 2017/00358; A61B 2017/0474

(57) ABSTRACT

The present disclosure relates to systems and methods for folding a leaflet. In an example, a catheter carrying a knot of a suture looped around a distal portion thereof, can be advanced into a central opening of a valvular structure, and a snare loop can be released from the catheter lumen to assume an expanded configuration distal to a leaflet of the valvular structure. A needle can is passed through the leaflet towards the snare loop, and a grasper is advanced through the needle to release a free end portion of the suture, extending from the knot. The snare loop can capture the suture as it is pulled into the catheter lumen, and a push shaft releases the knot from the catheter to form a loop around part of the host leaflet, which can be then reduced in size.

### CROSS-REFERENCE TO RELATED APPLICATIONS

This application is a continuation of International Application No. PCT/US2024/055891, filed Nov. 14, 2024, which claims the benefit of U.S. Provisional Application No. 63/600,551, filed Nov. 17, 2023, which is incorporated by reference herein.

### FIELD

The present disclosure relates to prosthetic valves, and to methods and systems for folding and/or capturing leaflets of existing valvular structures prior to or during implantation of a guest prosthetic valve to maintain access to one or more of the coronary arteries in the aorta.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient's vasculature (for example, through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve (“guest valve”) is delivered to the heart in a crimped state, as described above for the “native” TAVR. The guest valve is positioned within the previously implanted prosthetic valve (“host valve”) and then expanded to its functional size. The host valve in a ViV TAVR procedure can be a surgically implanted prosthetic valve or a transcatheter prosthetic valve. The term “host valve” is also used herein to refer to the native aortic valve in a native TAVR procedure.

### SUMMARY

Needles or other perforating tools can be utilized for piercing existing leaflets to form an opening that modifies the existing valvular structure, after which a guest prosthetic valve can be implanted in the modified valvular structure, mitigating the risk of coronary ostial obstruction. The leaflet is a relatively thin tissue having a free edge opposite to attachment on an opposite end to the aortic wall in the case of a native leaflet of an aortic valve, or to a frame of a previously implanted prosthetic valve in the case of ViV procedures. Perforation of the leaflet by pushing a needle or other perforating tool there-against, can move the leaflet to some extent due to the push force applied thereto. Even if the needle or other tool does puncture eventually through the tissue material, such initial movement can lead to the penetration point being in a different region of the leaflet relative to the initial point of contact.

According to some aspects of the disclosure, there is provided a leaflet folding system comprising a catheter defining a catheter lumen, a snare device axially movable through the catheter lumen.

In some examples, the leaflet folding system further comprises a needle defining a needle lumen.

In some examples, the leaflet folding system further comprises a grasper axially movable through the needle lumen.

In some examples, the leaflet folding system further comprises, and a suture.

In some examples, the suture comprises a knot looped around a distal portion of the catheter.

In some examples, the suture further comprises a first portion extending proximally from the knot, and a second portion extending from the knot towards the grasper.

In some examples, the leaflet folding system further suture comprises a push shaft disposed around the catheter and axially movable relative to the catheter.

In some examples, the second portion comprises a free end portion and is releasably attached to the grasper at the free end portion.

In some examples, the push shaft is configured to release the knot from the distal portion of the catheter by axially moving at least one of the push shaft and the catheter relative to the other, while the knot is engaged by the push shaft.

In some examples, the grasper comprises a grasper shaft and a grasper head at a distal end of the grasper shaft.

In some examples, the grasper head is configured to transition between a closed configuration and an open configuration.

In some examples, the grasper head is configured to release the free end portion of the suture in the open configuration.

In some examples, the snare device comprises a snare shaft and a snare loop at a distal end of the snare shaft.

In some examples, the snare loop is configured to transition between a compacted configuration and an expanded configuration.

In some examples, the snare loop is configured to transition to the expanded configuration when pushed distally out of the catheter lumen.

In some examples, the needle comprises a piercing tip configured to penetrate through a leaflet of a host valvular structure.

According to some aspects of the disclosure, there is provided a method comprising positioning a distal portion of a catheter inside a central opening of a host valvular structure.

In some examples, the method further comprises exposing a snare loop out of the catheter by advancing a snare device comprising the snare loop through a catheter lumen of the catheter, thereby causing the snare loop to assume an expanded configuration thereof.

In some examples, the method further comprises forming a puncture on a host leaflet of the host valvular structure using a needle comprising a needle lumen, thereby forming a puncture through the host leaflet.

In some examples, the method further comprises distally advancing the needle through the puncture of the host leaflet and through an opening defined by the snare loop, to position a needle head of the needle distal to the snare loop.

In some examples, the method further comprises advancing a grasper out of the needle lumen, wherein the grasper comprises a grasper head to which a free end portion of a suture is attached.

In some examples, the grasper is in a closed configuration when positioned in the needle lumen.

In some examples, the method further comprises releasing the free end portion of the suture from the grasper head, such that the suture extends through the opening defined by the snare loop.

In some examples, the method further comprises capturing the suture by the snare loop and pulling the suture into the catheter lumen, by proximally pulling the snare device to re-insert the snare loop into the catheter lumen.

In some examples, the method further comprises releasing a knot which is looped around the distal portion from the catheter by applying a distally oriented force by a push shaft disposed around the catheter.

In some examples, the releasing the knot from the catheter further comprises forming a loop of the suture.

In some examples, the loop comprises a portion of the suture extending from the knot towards a free end of the host leaflet, bent around the free end and extending therefrom towards and through the puncture of the leaflet, and extending from the puncture towards the knot.

In some examples, the method further comprises, after the forming the loop, reducing the size of the loop, thereby compacting a part of the leaflet disposed inside the loop between the puncture and the free end.

In some examples, the releasing the knot comprises distally pushing the push shaft relative the catheter.

In some examples, the releasing the knot comprises proximally pulling the catheter relative to the push shaft.

In some examples, the method further comprises, after the cutting, mounting a guest prosthetic valve within the host valvular structure, wherein the compacted part of the host leaflet allows blood to flow through a frame of the guest prosthetic valve to a coronary artery, which would otherwise be blocked by the host leaflet of the host valvular structure.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 is a cross-sectional view of a native aortic valve.

FIG. 2A shows a cross-sectional view of a prosthetic heart valve implanted in the native aortic valve of FIG. 1, according to an example.

FIG. 2B shows the implanted prosthetic heart valve of FIG. 1A as viewed from the ascending aorta, according to an example.

FIG. 3 shows a valve-in-valve implantation within the native aortic valve of FIG. 1, according to an example.

FIG. 4A shows an exemplary leaflet folding system positioned at a host valvular structure.

FIG. 4B shows the system of FIG. 4A, with a snare device deployed out of a catheter of the system.

FIG. 4C shows the system of FIG. 4B, with a needle distally advanced to pierce through a host leaflet of the host valvular structure.

FIG. 4D shows the system of FIG. 4C, with a grasper advanced through the needle to release a free end portion of a suture from a grasper head thereof.

FIG. 4E shows the system of FIG. 4D, with the suture forming a loop around part of the host leaflet.

FIG. 4F shows the system of FIG. 4E, with the suture captured by the snare device pulled through the catheter to tighten the loop around the host leaflet.

FIG. 4G shows the system of FIG. 4F, with a knot of the suture released, by a push shaft, from a distal portion of the catheter.

FIG. 4H shows the system of FIG. 4G, with the loop tightened to fold the corresponding part of the host leaflet.

FIG. 4I shows the loop locked around the folded part of the host leaflet, with the system of FIG. 4H retracted from the host valvular structure.

FIG. 4J shows the suture of FIG. 4I cut at a portion proximal to the knot.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a”, “an”, and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner”, “outer”, “upper”, “lower”, “inside”, “outside”, “top”, “bottom”, “interior”, “exterior”, “left”, right”, and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (for example, the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction”, “radial direction”, and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first”, “second”, and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a”, “b”, “c”, etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

In order to avoid undue clutter from having too many reference numbers and lead lines on a particular drawing, some components will be introduced via one or more drawings and not explicitly identified in every subsequent drawing that contains that component.

for folding and/or capturing leaflets of a heart valve to avoid, or at least reduce the risk of, obstructing blood flow to the coronary arteries. Prior to or during implantation of a prosthetic heart valve within an existing valvular structure, a part of a host leaflet (or parts of host leaflets) can be folded upon itself and held distal to the coronary ostia by one or more sutures. In some examples, the existing valvular structure can be a native aortic valve (for example, normal or abnormal, such as bicuspid aortic valve (BAV)) or a prosthetic valve previously implanted in the native aortic valve. When a new prosthetic heart valve is subsequently installed within the existing valvular structure, the captured and/or folded leaflets of the existing valvular structure are disposed at locations distal to the coronary artery ostia, thereby allowing blood to flow unobstructed to the coronary arteries. While described with respect to aortic valve, it should be understood that the disclosed examples can be adapted to deliver devices that can modify an existing valvular structure in any of the native annuluses of the heart (for example, the aortic, pulmonary, mitral, and tricuspid annuluses), and can be used with any of various delivery approaches (for example, retrograde, antegrade, transseptal, transventricular, transatrial, etc.).

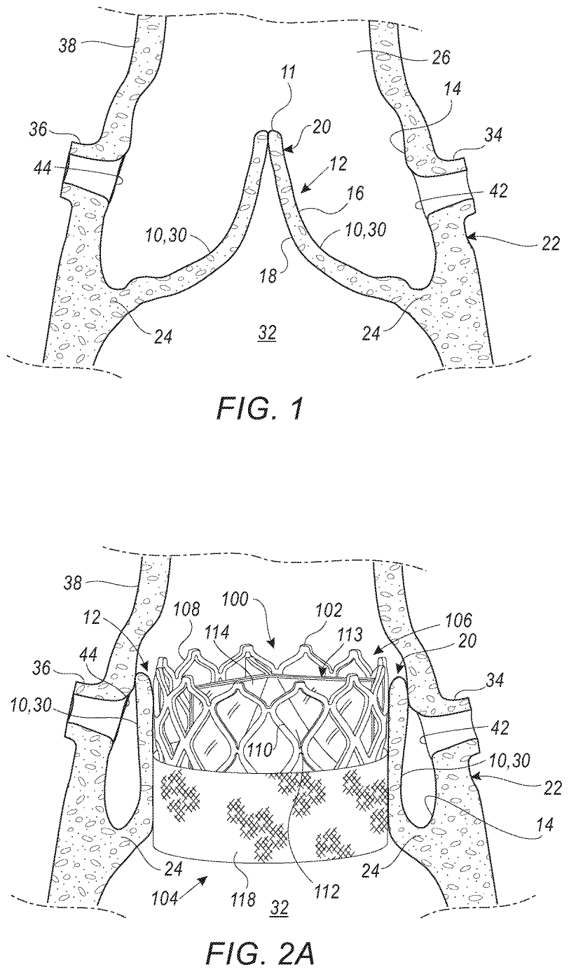

FIG. 1 illustrates an anatomy of the aortic root 22, which is positioned between the left ventricle 32 and the ascending aorta 26. The aortic root 22 includes a native aortic valve 20 having a native valvular structure 29 comprising a plurality of native leaflets 30. Normally, the native aortic valve 20 has three leaflets (only two leaflets are visible in the simplified illustration of FIG. 1), but aortic valves with fewer than three leaflets are possible. The leaflets 30 are supported at native commissures by the aortic annulus 24, which is a ring of fibrous tissue at the transition point between the left ventricle 32 and the aortic root 22. The leaflets 30 can cycle between open and closed positions (the closed position is shown in FIG. 1) to regulate flow of blood from the left ventricle 32 to the ascending aorta 26, wherein free edges 11 of the leaflets move towards each other to coapt in the closed state, and away from each other in the open state. Branching off the aortic root 22 are the coronary arteries 34, 36. The coronary artery ostia 42, 44 are the openings that connect the aortic root 22 to the coronary arteries 34, 36. Each leaflet 10, 30 can define a first surface 16 which is a proximally facing surface, such as by being oriented towards the ascending aorta 26 and/or coronary artery ostia 42, 44 in the case of leaflets 30 of an aortic valve 20, and a second surface 18 which is a distally facing surface, such as by being oriented towards the left ventricle 32 for leaflets 30 of an aortic valve 20.

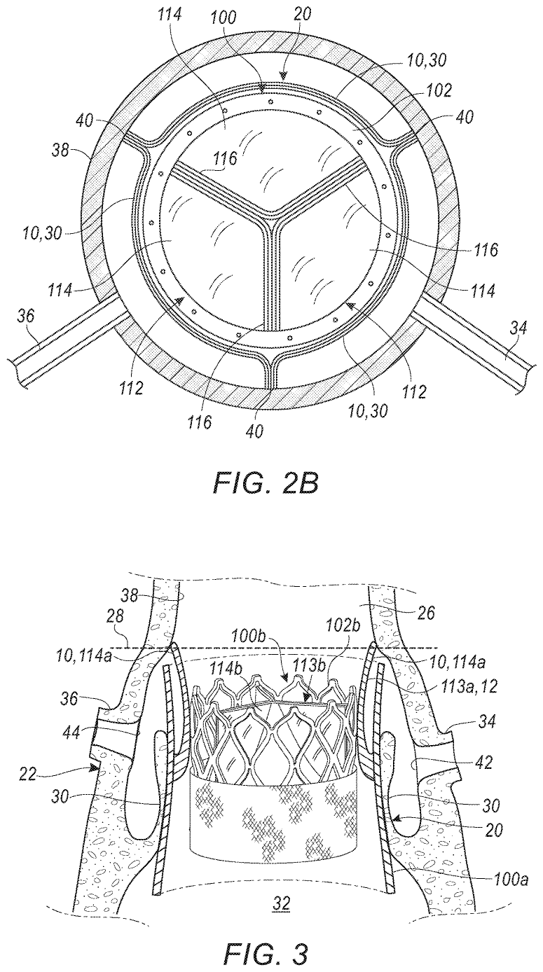

FIGS. 2A-2B show an exemplary prosthetic valve 100 that can be implanted in a native heart valve, such as the native aortic valve 20 of FIG. 1. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus (not shown) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/085,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 2A-2B show an example of a prosthetic valve 100, which can be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can be made of various suitable materials, including plastically-deformable materials such as, but not limited to, stainless steel, a nickel based alloy (for example, a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can be made of shape-memory materials such as, but not limited to, nickel titanium alloy (for example, Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 is an annular, stent-like structure comprising a plurality of intersecting struts 108. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in U.S. Pat. No. 9,155,619, which is incorporated herein by reference.

The struts 108 can include a plurality of angled struts and vertical or axial struts. At least some of the struts 108 can be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 113 of the prosthetic valve 100 can include a plurality of prosthetic valve leaflets 114 (for example, three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 114 can be arranged together to form prosthetic valve commissures 116 that are coupled (directly or indirectly) to respective portions of the frame 102, thereby securing at least a portion of the valvular structure 113 to the frame 102. The prosthetic valve leaflets 114 can be made from, in whole or part, biological material (for example, pericardium), bio-compatible synthetic materials, or other such materials. Further details regarding transcatheter prosthetic valves, including the manner in which leaflets 114 can be coupled to the frame 102 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

In some examples, the prosthetic valve 100 can comprise at least one skirt or sealing member. For example, the prosthetic valve 100 can include an inner skirt (not shown in FIG. 2A-2B), which can be secured to the inner surface of the frame 102. Such an inner skirt can be configured to function, for example, as a sealing member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner surface of frame 102, while the leaflets can be sutured to the inner skirt along a scalloped line (not shown). An inner skirt can be coupled to the frame 102 via sutures or another form of coupler.

The prosthetic valve 100 can comprise, in some examples, an outer skirt 118 mounted on the outer surface of frame 102 (as shown in FIGS. 2A-2B), configured to function, for example, as a sealing member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can be coupled to the frame 102 via sutures or another form of coupler.

Any of the inner skirt and/or outer skirt can be made of various suitable biocompatible materials, such as, but not limited to, various synthetic materials (for example, PET) or natural tissue (for example pericardial tissue). In some cases, the inner skirt can be formed of a single sheet of material that extends continuously around the inner surface of frame 102. In some cases, the outer skirt 118 can be formed of a single sheet of material that extends continuously around the outer surface of frame 102.

The cells 110, defined by interconnected struts 108, define cell openings 112. While some of the cell openings 112 can be covered by the inner skirt and/or the outer skirt, at least a portion of the cell opening 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. The prosthetic valve 100 is then radially expanded against the host leaflets 10. As illustrated, the host leaflets 10 form a tube around the frame 102 of the prosthetic valve 100 after the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. FIG. 3 illustrates an exemplary hypothetical coronary artery obstruction that could occur in some cases from implantation of a prosthetic valve 100b within a previously implanted prosthetic valve 100a (for example, after a ViV procedure). In this example, the prosthetic valve 100b is the guest valve or new valve, and the prosthetic valve 100a is the host valve or old valve. In this example, the prosthetic valve 100a was previously implanted within the orifice of the native aortic valve 20. Each of the prosthetic valves 100a, 100b can have the general structure of the prosthetic valve 100 described with reference to FIGS. 2A-2B, though in some examples, each of the prosthetic valves 100a, 100b can be a different type of prosthetic valve. For example, a balloon expandable guest valve 100b can be implanted inside a previously implanted mechanically expandable or self-expandable host valve 100a.

During implantation of the prosthetic valve 100b, the prosthetic valve 100b is positioned within a central region defined between the leaflets 114a of the prosthetic valve 100a, which now take the role of host leaflet 10. The prosthetic valve 100b is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary artery ostia 42, 44, causing coronary artery ostia obstruction. Alternatively, the guest prosthetic valve 100b can displace the host leaflets 114a outwardly against the frame 102a of the host valve 100a, thereby blocking the flow of blood through the frame 102a to the coronary ostia 42, 44.

In some patient anatomies (for example, when the outflow end 106 of the prosthetic valve 100 is at the STJ level 28 and the diameter of the prosthetic valve 100 is similar to the STJ diameter such that the frame 102 touches or is very close to the aortic wall 38 at the STJ level 28), the host leaflets 10 may compromise the ability for future access into the coronary arteries 34, 36 or perfusion through the frame 102 to the coronary arteries 34, 36 during the diastole phase of the cardiac cycle. Similar problems may occur in some patient anatomies either when a guest prosthetic valve 100b is percutaneously expanded within a previously implanted host prosthetic valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary artery ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 typically sits lower than the right coronary ostium 44.

The term “host valve” as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can be a surgically implanted prosthetic heart valve (known as a “surgical valve”) or a transcatheter heart valve. The term “guest valve”, as used herein, refers to a prosthetic valve implanted in a host valve, which can be either a native heart valve or a previously implanted prosthetic valve. Similarly, the term “host leaflets 10”, as used herein, refers to native leaflets 30 of a native valve in which a new guest prosthetic valve 100 is implanted, or to prosthetic valve leaflets 114a of a previously implanted host valve 100a in which a new guest prosthetic valve 100b is implanted.

To avoid obstruction of blood flow to the coronary arteries 34, 36, the valvular structure 12 of the existing host valve (whether a native aortic valve or a previously implanted prosthetic valve) can be modified by components of a leaflet folding system prior to or during implantation of a new prosthetic valve within the existing valvular structure 12. In some examples, the host valvular structure 12 is modified by folding a part of a host leaflet 10 (or parts of host leaflets 10) upon itself using the leaflet folding system. The modification captures the folded leaflet in a position that avoids obstructing the corresponding coronary ostium, thereby allowing blood to flow to the coronary arteries 34, 36.

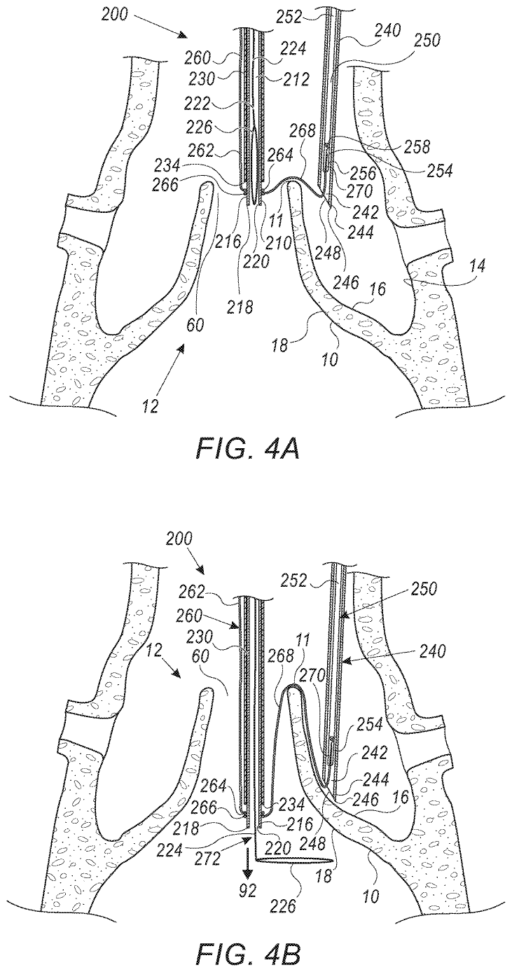

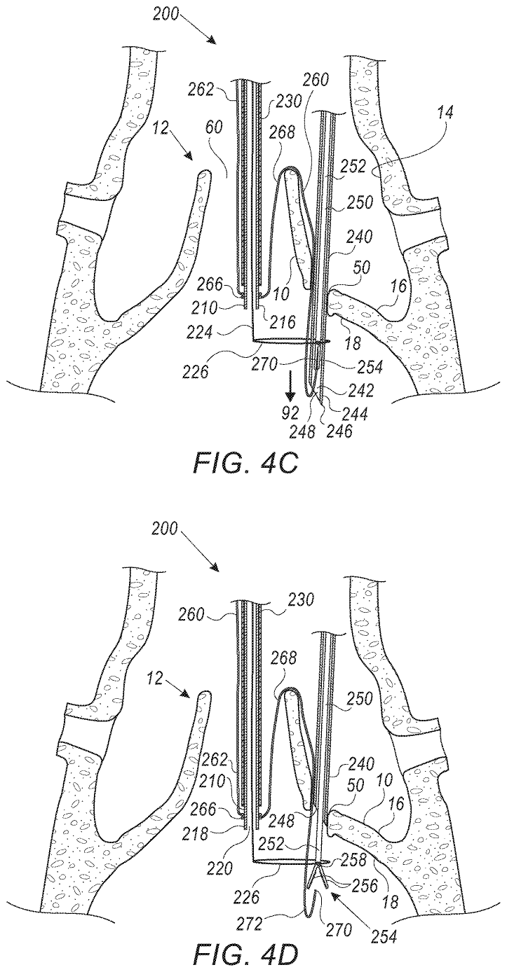

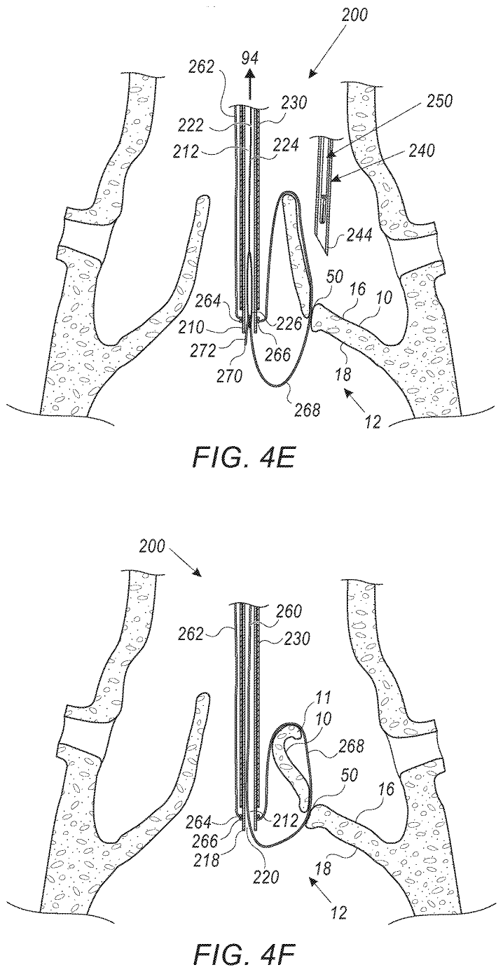

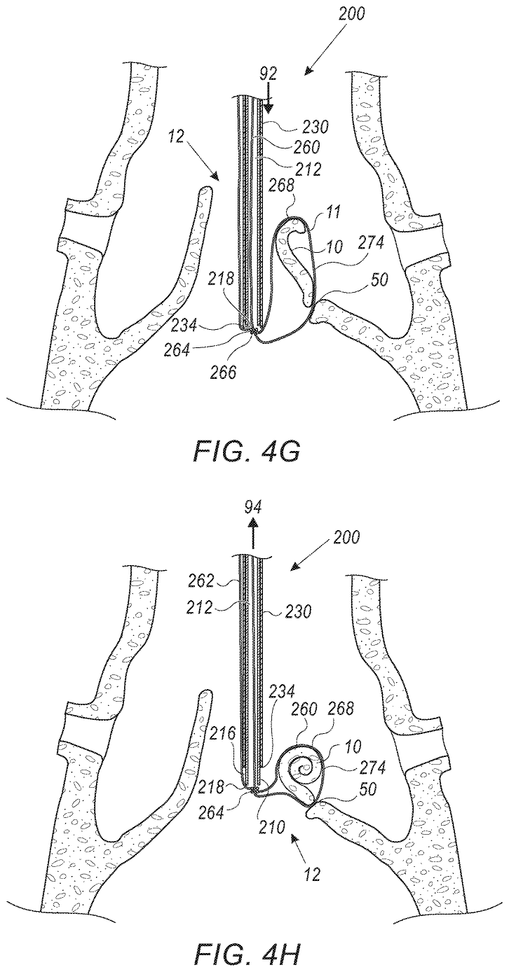

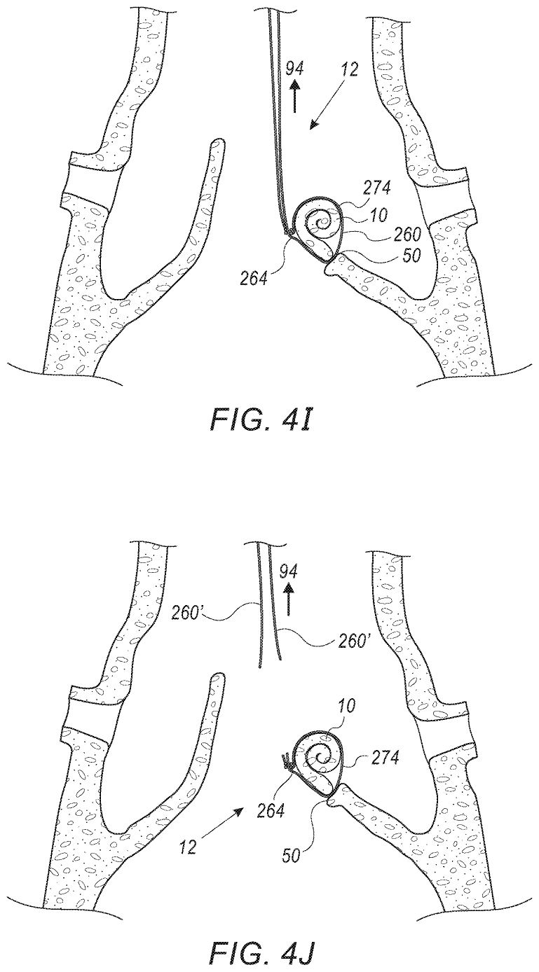

FIGS. 4A-4J illustrate some steps in a method for utilizing a system 200 for folding and/or capturing a host leaflet 10, which can be performed prior to implanting a guest prosthetic valve inside the host valvular structure 12. The leaflet folding system 200 can be used to fold and/or capture part of a host leaflet 10, such as a native leaflet 30 or a prosthetic valve leaflet 114 of a previously implanted prosthetic valve. The terms “leaflet folding system 200” and “system 200”, as used herein, are interchangeable.

The leaflet folding system 200 includes a catheter 210 defining a catheter lumen 212, a snare device 222 extendable through the catheter lumen 212, a push shaft 230 disposed around the catheter 210, a suture 260 having a knot 264 looped around a distal portion 216 of the catheter 210, a hollow needle 240 defining a needle lumen 242, and a grasper 250 extendable through the needle lumen 242. The snare device 222 and the catheter 210 are axially movable relative to each other. The push shaft 230 and the catheter 210 are axially movable relative to each other. The grasper 250 and the needle 240 are axially movable relative to each other. In some examples, the catheter 210 and the needle 240 can be independently movable relative to each other in the axial direction and/or other directions.

Needle 240 comprises a needle head 244 that can define an angled surface, terminating at a piercing tip 246 to facilitate piercing of a host leaflet 10 when the needle 240 is pressed against the leaflet. The needle head 244 is affixed or integrally formed with the needle 240, and is not configured to detach therefrom during the leaflet folding procedure. The needle lumen 242 is sized to allow passage of the grasper 250 therethrough, wherein the grasper 250 can be axially movable inside needle lumen 242, and can be either completely concealed inside the needle lumen 242, or have at least a distal portion thereof extending distally through a needle opening 248 defined by the needle head 244 at the distal end of the needle lumen 242.

Catheter 210 comprises a distal portion 216 terminating at a distal end 218. The catheter lumen 212 is sized to allow passage of the snare device 222 therethrough, wherein the snare device 222 can be axially movable inside catheter lumen 212, and can be either completely concealed inside the catheter lumen 212, or have at least a distal portion thereof extending distally through a catheter opening 220 defined by the distal end 218 at a distal end of the catheter lumen 212. The push shaft 230 can define a lumen sized to allow passage of the catheter 210 therethrough. The push shaft 230 can be in the form of an overtube, disposed over, and axially movable relative to, the catheter 210.

The suture 260 include a knot 264 defining one or more loop(s) 266 that can be looped around the distal portion 216, and can be axially slid over the distal portion 216. The suture 260 comprises a first portion 262 extending proximally from the knot 264, such as over a length of, and parallel to, the catheter 210. The suture 260 further comprises a second portion 268, extending from the knot 264 towards a free end portion 270 of the suture, wherein the free end portion 270 is configured to be releasably grasped by the grasper 250.

The grasper 250 comprises a grasper shaft 252 extending through and along a length of the needle 240, and a grasper head 254 at a distal end of the grasper shaft 252. The grasper head 254 can include jaws 256 which are pivotably movable to each, connected at a hinge 258 that can be in the form of a pin, or any other type of hinge configured to allow for rotational movement of at least one of the jaws 256 relative to at least one other jaw 256. While a grasper head 254 equipped with two jaws 256 is shown in the exemplary system 200 illustrated in FIGS. 4A-4E, it is to be understood that in some examples, more than two jaws can be provided. The grasper head 254 can be an atraumatic portion of the grasper 250, for example by including jaws 256 that have relatively blunt distal ends.

The distal end portion of the system 200, which can include the distal portion 216 and the needle head 244, is configured to be inserted into a patient's vasculature, such as within an ascending aorta, and to be advanced towards the host valvular structure 12. FIG. 4A shows the system 200 positioned at the host valvular structure 12 such that the distal end 218 of the catheter 210 is facing or is positioned at a region of the host valvular structure 12 between the host leaflet 10. In some examples, the distal end 218 can be oriented towards, or be positioned inside, a central opening 60 of the host valvular structure 12, defined as the opening defined between the leaflets 10 and/or free edges 11 thereof, in the open state of the leaflets. While the distal portion 216 is directed towards and/or into the central opening 60 defined between the leaflet 10, the needle head 244 is oriented towards a host leaflet 10, facing the first surface 16 of the leaflet 10 as illustrated in FIG. 4A.

In the example illustration of the system 200 shown in FIG. 4A, knot 264 is looped around the distal portion 216 in a manner that is tight enough to prevent the knot 264 from being spontaneously slid from around the catheter during delivery towards the host valvular structure 12, and the push shaft distal edge 234 is proximal to the distal end 218 and the knot 264. In some examples, the first portion 262 can extend distally from a handle (not shown) of the system 200 towards the knot 264 along a length of the catheter 210, optionally disposed outside of the catheter 210, and optionally disposed outside of the push shaft 230.

The grasper head 254 is configured to transition between a closed configuration in which the jaws 256 are pressed against each other, as illustrated for example in FIGS. 4A-4C, and an open configuration in which the jaws 256 extend away from each other, as illustrated for example in FIG. 4D. The needle lumen 242 can be sized to maintain the grasper head 254 in a closed configuration when the grasper head 254 is positioned therein. In the example illustration of the system 200 shown in FIG. 4A, the grasper head 254 is positioned inside the needle lumen 242, proximal to the needle opening 248, and a portion of the suture 260, optionally including the free end portion 270, is tightly grasped between the jaws 256 of the grasper head 254, such that the free end portion 270 can also reside, partially or completely, inside the needle lumen 242, proximal to the needle opening 248.

As further shown in FIG. 4A, the system 200 can be advanced into the host valvular structure 12, such that the distal portion 216, having the knot 264 looped therearound, is disposed on one side of the host leaflet 10, such as facing the second surface 18 of the leaflet 10 when the distal portion 216 is positioned in the central opening 60, while the needle head 244 is disposed on the opposite side of the host leaflet 10, facing the first surface 16 of the leaflet 10. The second portion 268 extends from the knot 264 towards and into the needle 240, entering through the needle opening 248 into the needle lumen 242, having its free end portion 270 grasped by the grasper head 254 between the jaws 256. The second portion 268 can be supported by, or be position proximal to, the free edge 11 of the host leaflet 10, at a section of the suture 260 between the knot 264 and the grasper head 254.

It is to be understood that any reference to a free end portion 270 being grasped by the grasper head 254 or released therefrom, throughout the specification and the claims, is not limited to the terminal end of the suture 260 being positioned between, or released from between, the jaws 256, but is also meant to cover examples in which a distal end portion of the suture 260, leading to the terminal end of the suture, can be grasped or released from between the jaws 256, while the suture's terminal end itself may be positioned sideways from the jaws 256 and not necessarily held therebetween, such as when a short slack of suture 260 extends from between the jaws 256 to the suture's terminal end.

The snare device 222 includes a snare shaft 224 and a snare loop 226 at the distal end of the snare shaft 224. The snare loop 226 is configured to transition between a compacted configuration, as illustrated for example in FIG. 4A, and an expanded configuration, as illustrated for example in FIG. 4B. In the example illustration of the system 200 shown in FIG. 4A with the distal end portion of the system 200 positioned in the host valvular structure 12, the snare loop 226 is in the compacted configuration, having a generally elongated narrow configuration with opposite sides thereof maintained in close proximity to each other, such that the snare device 222 with the snare loop 226 can be axially advanced or retracted inside catheter lumen 212.

The catheter lumen 212 can be sized to maintain the snare loop 226 in a compacted configuration, while the snare loop 226 is positioned inside catheter lumen 212. The snare loop is shown to be positioned inside the catheter lumen 212, proximal to the distal end 218, in the position illustrated in FIG. 4A. In some examples, the snare device 222 can be disposed inside the catheter lumen 212, with the snare loop 226 positioned proximal to distal end 218, during delivery of the system 200 towards the host valvular structure 12. In some examples, the snare loop 226 can be disposed at a more proximal region of the catheter 210, or even completely out of the catheter lumen 212, during advancement of the catheter 210 through the patient's vasculature, and distally advanced through the catheter lumen 212, or inserted into and then advanced through the lumen 212, closer to the distal end 218 (as shown, for example, in FIG. 4A), when the distal portion 216 reaches the host valvular structure 12.

In some examples, the catheter 210 and the needle 240 can be simultaneously advanced through a patient's vasculature, towards the host valvular structure 12. In some examples, the needle 240 and the grasper 250 can be simultaneously advanced through a patient's vasculature, towards the host valvular structure 12. In some examples, the catheter 210 and the snare device 222 can be simultaneously advanced through a patient's vasculature, towards the host valvular structure 12. In some examples, the catheter 210, the needle 240, and the grasper 250, can be simultaneously advanced through a patient's vasculature, towards the host valvular structure 12. In some examples, the catheter 210, the snare device 222, the needle 240, and the grasper 250, can be simultaneously advanced through a patient's vasculature, towards the host valvular structure 12.

When the distal end portion of the system 200 approaches the host valvular structure 12, the catheter 210 can be distally advanced in the direction indication by arrow 92 in FIG. 4B, into the central opening 60 of the valvular structure 12, between the host leaflets 10, such that the distal portion 216 and the knot 264 looped therearound are positioned distal to the free edge 11 of the host leaflet 10, while the needle 240 can be distally advanced also in the direction of arrow 92 past the free edge 11 of the leaflet 10, towards, and optionally up to contact with, the host leaflet 10. In this configuration, illustrated in FIG. 4B, part of the host leaflet 10, leading to the free edge 11, extends between the catheter 210 and the needle 240, such that the distal portion 216 and the knot 264 looped therearound are disposed on one side of the host leaflet 10, facing its second surface 18, while the needle head 244 is disposed on the other side of the leaflet 10, facing its first surface 16.

In this configuration, the second portion 268 can proximally extend from the knot 264 towards the free edge 11 of leaflet 10, bend over the free edge 11, and extend distally therefrom towards the needle head 244, at which point it enters through needle opening 248 into the needle lumen 242, towards the grasper head 254. In some examples, the catheter 210 and the needle 240 can be simultaneously advanced to the position shown in FIG. 4B. In some examples, the catheter 210 and the needle 240 can be sequentially advanced one after the other, such as by advancing the needle 240 towards the first surface 16 after advancing the catheter 210 into the central opening 60, or by advancing the catheter 210 into the central opening 60 after advancing the needle 240 towards the first surface 16.

With the distal portion 216 positioned distal to the free edge 11 of host leaflet 10, the snare device 222 can be distally advanced so as to expose the snare loop 226 out of catheter lumen 212, allowing the snare loop 226 to assume its expanded configuration when positioned distal to the catheter opening 220. In some examples, the snare loop 226 is made of a shape-memory material, such as Nitinol, which is shape-set to assume its functional or expanded configuration when not restricted by an external restricting component, such as the catheter 210. The snare loop 226 defines, at least in the expanded configuration, an opening 228, through which a suture, such as suture 260, can extend.

In some examples, the snare loop 226 can be bendable relative to the snare shaft 224, and can be axially aligned with the snare shaft 224 when retained, in the compacted configuration, inside the catheter lumen 212, as shown for example in FIG. 4A, yet configured to assume an angled configuration by which a plane defined by the snare loop 226 is angled relative to the snare shaft 224, as shown for example in FIG. 4B. When formed of a shape-memory material, such as Nitinol, the snare loop 226 can be shape-set to assume the bent configuration, relative to the snare shaft 224, when not restricted by an external restricting component, such as the catheter 210. In some examples, the snare loop 226 is configured to extend from the snare shaft 224 at an angle less than 180 degrees, such as a 90-degree angle illustrated in FIG. 4B, when in the expanded configuration. An angled orientation of the snare loop 226 can advantageously position the opening 228 defined by the snare loop 226 distally to the second surface 18 of the leaflet 10, optionally in alignment with the needle head 244.

The hollow needle 240 can be then distally advanced in the direction indicated by arrow 92, to puncture the host leaflet 10 and form a puncture 50 within host leaflet 10, as shown in FIG. 4C. The needle 240 is oriented towards the opening 228 of the snare loop 226 as it is passed through the host leaflet 10, such that further advancement thereof in the distal direction 92 will cause the needle to extend through the opening 228, positioning the needle head 244 distal to the snare loop 226.

In some examples, the snare loop 226 can be exposed out of the catheter lumen 212 and positioned, in its expanded configuration, distal to the second surface 18 of the leaflet 10, while the needle 240 is still kept proximal to the leaflet 10, after which the needle 240 can pierce through the leaflet 10 and extend further through the opening 228 of the snare loop 226, as shown in the exemplary sequence illustrated in FIG. 4B-4C. In some examples, the needle 240 can be distally advanced to pierce through the host leaflet 10 and form a puncture 50 in the leaflet 10, while the snare loop 226 can be kept in a compacted configuration inside the catheter lumen 212, after which the snare loop 226 can be exposed out of the catheter lumen 212 and positioned, in its expanded configuration, distal to the second surface 18 of the leaflet 10 and the needle head 244. The needle 240 can be further advanced, in such examples, through the opening 228, so as to positioning the needle head 244 distal to the snare loop 226.

In some examples, the needle 240 is not necessarily aligned with or oriented towards the snare loop 226 during penetration into leaflet 10 to form the puncture 50, and at least one of the needle 240 or the snare device 222 can be then maneuvered by an operator of the system 200, to oriented the needle 240 towards the opening 228 of the snare loop 226 prior to further distal advancement of the needle 240.

As further shown in FIG. 4D, the grasper head 254 can be then exposed through the needle opening 248 and out of the needle lumen 242, allowing it to assume an open configuration by which the jaws 256 pivot away from each other, thus releasing the suture 260 with the free end portion 270 positioned distal to the snare loop 226. In some examples, the grasper head 254 is exposed by being distally pushed out of the needle lumen 242 through the needle opening 248. Since the needle head 244 and needle opening 248 are positioned distal to the snare loop 226 prior to extension of the grasper 250, further distal advancement of the grasper will cause the grasper head 254 to be also positioned distal to the snare loop 226, such that when the suture 260 is released therefrom, it extends through snare opening 228.

In some examples, the grasper head 254 is exposed by proximally retracting the needle 240 relative to the grasper head 254, allowing the jaws 256 to extend away from each other as soon as the needle opening 248 is pulled proximal to the hinge 258. In such examples, not only will the needle head 244 be positioned distal to the snare loop 226, but the grasper head 254, still retained in a closed configuration inside the needle 240, will be also positioned distal to the snare loop 226, prior to exposing the grasper head 254, such that when the needle 240 is proximally pulled to expose the grasper head 254, the grasper head will necessarily be positioned distal to the snare loop 226 as it transitions to the open configuration and releases the suture 260 in a manner that similarly results in the suture 260 extending through the opening 228. In some examples, the grasper head 254 is exposed by simultaneously pushing the grasper 250 in the distal direction 92 and proximally pulling the needle 240.

In some examples, the needle lumen 242 has a diameter that is equal to or less than twice the thickness of a jaw 256 and the thickness of the suture 260, such that when the grasper head 254 is retained in a closed configuration inside the needle lumen 242, the diameter of the needle lumen 242 will cause the jaws to pinch the free end portion 270 therebetween. The distal ends of the jaws 256 can define a gap therebetween, in the open configuration of the grasper head 254, that is greater than the thickness of the suture 260, such that when the jaws 256 are deflected away from each other, the suture 260 can be released from therebetween. The diameter of the needle lumen 242 can be less than the gap formed between the jaws 256 in the open configuration, and/or less than the gap combined with twice the thickness of a jaw 256, which will allow the jaws 256 to extend away from each other when released from the needle lumen 242.

In some examples, the jaws 256 can be biased to the open configuration of the grasper head 254. In some examples, a torsion spring or other biasing member can be utilized to bias the jaws 256 away from each other in a free or unconstrained configuration of the grasper head 254. In some examples, the hinge 258 is implemented as an integrally formed pivot point, such as a live hinge, without including separate components such as a pin, wherein the jaws 256 can be formed of a shape-memory material, such as Nitinol, which can be shape-set to assume the outwardly biased configuration of the jaws 256 in a free state thereof. In some examples, the jaws 256 are not necessarily biased to the open configuration, but can be actively transitioned, such as by a pull wire or other actuation mechanisms, between a closed configuration and an open configuration.

After releasing the suture 260 from the grasper head 254, the needle 240 and the grasper 250 can be pulled in a proximal direction 94 out of host leaflet 10, leaving the second portion 268 extending through the puncture 50 formed in the host leaflet 10. In some examples, the grasper 250 can be pulled so as to position the grasper head 254 in a closed configuration, back within the needle lumen 242, prior to and/or during proximal retraction of the needle 240 out of the host leaflet 10. In some examples, the needle 240 and the grasper 250 can be simultaneously pulled away from the host leaflet 10, at least after re-concealing the grasper head 254 inside the needle lumen 242.

With the suture 260 extending through the opening 228, the snare device 222 can be retracted in a proximal direction 94 so as to pull the snare loop 226 back into the catheter lumen 212, with a portion of the suture 260 captured by the snare loop 226 as it transitions back to the compacted configuration. In some examples, the snare loop 226 can be formed of a wire having a thickness that can be also referred to as a snare loop wire thickness. The catheter lumen 212 can have a diameter that is equal to or less than twice the snare loop wire thickness combined with the thickness of the suture 260, such that when the snare loop 226 is re-concealed inside the catheter lumen 212, the diameter of the catheter lumen 212 will cause both sides of the snare loop 226 to pinch the portion of the suture 260 that extends through the opening 228, therebetween.

As further shown in the configuration illustrated in FIG. 4E, this results in the second portion looping around part of the host leaflet 10 extending between the puncture 50 and the free edge 11, wherein the second portion 268 extends from the suture 260 disposed around the distal portion 216 towards the free edge 11, bend over the free edge 11 to extend therefrom towards and through the puncture 50, and finally extending from the puncture 50 back towards the distal portion 216. The portion of the suture 260 captured by the compacted snare loop 226 can be the free end portion 270, optionally defining a suture slack 272 further extending from the snare loop 226 to the terminal end of the suture 260.

In some examples, retraction of the needle 240 and/or grasper 250 from the host leaflet 10 is performed prior to retraction of the snare loop 226 back into the catheter lumen 212. In some examples, retraction of the snare loop 226 back into the catheter lumen 212 is performed prior to retraction of the needle 240 and/or grasper 250 from the host leaflet 10. In some examples, the needle 240 and/or grasper 250 can be simultaneously retracted with the snare device 222. Retraction of the needle 240 and/or grasper 250 from the host leaflet 10 can include, in some examples, retrieval of the needle 240 and/or grasper 250 from the patient's body.

While not shown in the illustrated example, it is to be understood that in some examples, an additional tube, such as an overtube, can be disposed over the needle 240. In such cases, the needle 240 and the overtube can be axially movable relative to each other. The needle 240 can be delivered towards the host valvular structure 12 with the needle head 244 retained inside the overtube, thereby concealing the piercing tip 246 to prevent it from accidentally damaging anatomical structures during advancement towards the site of treatment. Once approaching the host valvular structure 12, the needle head 244 can be exposed by distally advancing the needle 240 relative to the overtube, by proximally pulling the overtube relative to the needle 240, or both.

Optionally, after re-insertion of the snare loop 226 back into the catheter lumen 212, the snare device 222 can be further retracted to proximally pull the free end portion 270 farther away from the catheter opening 220, which can tighten the loop formed by the second portion 268 around the corresponding part of the host leaflet 10, as illustrated in FIG. 4F.

With the suture 260 grasped by the snare loop 226, so as to extend into the catheter lumen 212 through the catheter opening 220, the push shaft 230 can be distally slid over the catheter 210, so as to push the knot 264, in the distal direction 92, to a position distal to the distal end 218, placing the loop(s) 266 of the knot 264 around the portion of the suture 260 extending from the puncture 50 into the catheter lumen 212. The knot 264 can define a knot thickness that can depend on the number of its loops 266 and their arrangement, or other characteristics of the knot 264 such as the type of knot and the manner by which it is formed, wherein the knot thickness is defined as the dimension of the knot 264 measured between an inner edge thereof, which is in contact with the distal end 218, and an outer edge thereof, opposite to the outer surface of the catheter 210.

The inner diameter of the push shaft 230 and its thickness can be designed such that the push shaft distal edge 234 is aligned with the knot 264 and is congruent with at least part of the knot thickness, when the knot 264 is looped around the distal end 218, such that when the push shaft 230 is pushed in a distal direction 92, the push shaft distal edge 234 is configured to contact and push against the knot 264, pushing the knot 264 therewith in the distal direction 92, until the knot 264 is slid away and distally to the distal end 218.

Pushing the knot 264 to a position distal to the distal end 218 can be accomplished by distally pushing the push shaft 230 relative to the catheter 210, by proximally pulling the catheter 210 relative to the push shaft 230, or both. As shown in FIG. 4G, this result in a loop 274 defined by the suture 260 extending from the knot 264 towards the free edge 11, bent over the free edge 11 to extend therefrom towards and through the puncture 50, and finally extending from the puncture 50 back into the knot 264.

Further proximal retraction of the suture 260 through the knot 264 as shown in FIG. 4H, such as by additional pull force applied to the free end portion 270 captured by the snare loop 226, and/or by applying additional push force to the knot 264, can further tighten the loop 274 around the part of the host leaflet 10 retained therein. This can be performed while the first portion 262 is also proximally pulled, optionally simultaneously with the pulling of the portion of the suture extending through the knot 264. The amount of reduction in the size of the loop 274 can be chosen such that the folded part of the host leaflet 10 contained therein is positioned distal to the coronary artery ostium 42, 44, even when pressed radially outward by subsequent mounting of a guest prosthetic valve 100 within the host valvular structure 12. The knot is configured to tighten its loop(s) 266 around the portion of the suture 260 extending therethrough as the loop 274 is reduced in size, so as to lock the knot 264 in place when the loop 274 is no longer tightened.

Is some examples, the knot 264 is configured to self-tighten around the portion of the suture 260 extending through loop(s) 266 thereof as the suture 260 is pulled through the knot 264 and/or as the knot 264 is pushed relative to the portion of the suture 260 extending therethrough. In some examples, the knot 264 can be locked in placed by bonding, fusing, or encasing the suture portions forming the knot 264. In some examples, the knot 264 can be locked in place by a separate physical device (not shown) that can be brought into contact with the knot 264, for example provided as a tube or other member configured to slide over the portion of the suture 260 proximally extending from the knot 264. In some examples, the knot 264 can be tightened by proximally pulling the first portion 262 relative to the knot 264.

With the knot 264 locked in place, the catheter 210 and/or push shaft 230 can be proximally retracted away from the knot 264, and optionally retrieved from the patient's body, as illustrated in FIG. 4I, and the suture 260 can be cut, for example at a location proximal to a location of the knot 264, with the cut portion of the suture 260 retracted from the host valvular structure, as shown in FIG. 4J. The cutting of the suture 260 can be made by a cutting tool (not shown) provided from a distal end of system 200 or provided via a separate catheter in the ascending aorta 26. In some examples, part of the catheter 210 (such as distal end 218) or part of the push shaft 230 (such as push shaft distal edge 234) can include a cutting element for cutting the suture 260 (examples not shown).

The suture loop 274 thus folds part of host leaflet 10 and captures it distal to coronary artery 34, 36, thereby preventing, or at least reducing the risk of obstruction of the coronary artery ostium 42, 44. If additional capture/folding of one or more host leaflets 10 of the host valvular structure 12 is desired, for example, to prevent, or at least reduce the risk of, obstruction of the ostium of coronary artery, then the techniques of FIGS. 4A-4J can be repeated in a similar manner with respect to the next host leaflet 10 using the same leaflet folding system 200 or a different leaflet folding system 200. Although not discussed in detail above, manipulation and/or movement of the needle 240, grasper 250, catheter 210, snare device 222, push shaft 230, and/or suture 260, including knot 264, within the patient's anatomy, may be performed using any tools employed in laparoscopic and/or transcatheter heart surgeries, such as, but not limited to, knot pushers, suture cutters, manipulators, and the like.

When no further leaflet capture is desired, a guest prosthetic valve 100 in a crimped state can subsequently be advanced to the host valvular structure 12 with captured host leaflets 10. The guest prosthetic valve 100 is disposed within the host valvular structure 12 and expanded, such that the captured host leaflets 10 are disposed on an external surface of the frame 102 of guest valve 100. However, the locked suture loops 274 retain the captured host leaflets 10 in a location distal to the coronary arteries 42, 44, thereby allowing blood to flow from the outflow end 106 of the guest prosthetic valve 100 to the coronary arteries 42, 44 once the guest valve implantation is completed.

In some examples, capturing and/or folding of host leaflet 10 is a host valvular structure, as described in any of the above noted examples, can be performed as part of, before, or after a balloon annular valvuloplasty procedure on the host valvular structure 12, for example, to prepare an host existing valve (such as a native aortic valve 20 or previously implanted prosthetic valve 100a) for subsequent implantation of a guest prosthetic valve 100b. capturing and/or folding of host leaflet 10 is a host valvular structure, as described in any of the above noted examples, can be performed as part of or preceding a transcatheter aortic valve implantation (TAVI) or transcatheter aortic valve replacement (TAVR) procedure, for example, to replace a native aortic valve or to replace a failing prosthetic valve (e.g., ViV procedure). As mentioned above, while the examples of FIGS. 4A-4J specifically illustrate capture/folding of host leaflets in a native aortic valve, it should be readily understood that the same techniques can be applied to previously-implanted prosthetic valves, for example, prior to or simultaneous with the mounting of a guest prosthetic valve 100b within the previously-implanted prosthetic valve 100a. Accordingly, examples of the disclosed subject matter are not limited to the particular illustrations.

Any exemplary system 200 disclosed herein, and/or components thereof, can be sterilized (for example, with heat, radiation, and/or chemicals, etc.) to ensure they are safe for use with patients, and any of the exemplary methods herein can include sterilization of the associated system 200 and/or components thereof, as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Some Examples of the Disclosed Technology

Some examples of above-described subject matter are enumerated below. It should be noted that one feature of an example in isolation or more than one feature of the example taken in combination and, optionally, in combination with one or more features of one or more examples below are examples also falling within the disclosure of this application.

Example 1. A leaflet folding system comprising:
a catheter defining a catheter lumen;
a snare device axially movable through the catheter lumen;
a needle defining a needle lumen;
a grasper axially movable through the needle lumen;
a suture comprising:
   a knot looped around a distal portion of the catheter;
   a first portion extending proximally from the knot; and
   a second portion extending from the knot towards the grasper; and
a push shaft disposed around the catheter and axially movable relative to the catheter;
   wherein the second portion comprises a free end portion and is releasably attached to the grasper at the free end portion; and
   wherein the push shaft is configured to release the knot from the distal portion of the catheter by axially moving at least one of the push shaft and the catheter relative to the other, while the knot is engaged by the push shaft.

Example 2. The system of any example herein, particularly of example 1, wherein a distal edge of the push shaft is proximal to the knot.

Example 3. The system of any example herein, particularly of example 2, wherein the distal edge of the push shaft is configured to contact the knot when the push shaft is distally pushed relative to the catheter.

Example 4. The system of any example herein, particularly any one of examples 1 to 3, wherein the grasper comprises a grasper shaft and a grasper head at a distal end of the grasper shaft, and wherein the grasper head is configured to transition between a closed configuration and an open configuration.

Example 5. The system of any example herein, particularly of example 4, wherein the grasper head is in the closed configuration when the suture is attached thereto.

Example 6. The system of any example herein, particularly of example 4 or 5, wherein the grasper head is positioned inside the needle lumen when the suture is attached thereto.

Example 7. The system of any example herein, particularly any one of examples 4 to 6, wherein the grasper head is configured to transition to the open configuration when pushed distally out of the needle lumen.

Example 8. The system of any example herein, particularly any one of examples 4 to 7, wherein the grasper head is configured to release the free end portion of the suture in the open configuration.

Example 9. The system of any example herein, particularly any one of examples 4 to 7, wherein the grasper head comprises a plurality of jaws pivotably movable relative to each other.

Example 10. The system of any example herein, particularly of example 9, wherein the plurality of jaws comprises two jaws connected to each other at a hinge.

Example 11. The system of any example herein, particularly of example 10, wherein the hinge comprises a pin.

Example 12. The system of any example herein, particularly of example 9 or 10, wherein the needle lumen defines a diameter that is not greater than a combined thickness of the two jaws and the suture.

Example 13. The system of any example herein, particularly any one of examples 1 to 12, wherein the snare device comprises a snare shaft and a snare loop at a distal end of the snare shaft, and wherein the snare loop is configured to transition between a compacted configuration and an expanded configuration.

Example 14. The system of any example herein, particularly of example 13, wherein the snare loop is in the compacted configuration when positioned inside the catheter lumen.

Example 15. The system of any example herein, particularly of example 13 or 14, wherein the snare loop is positioned inside the catheter lumen during delivery of the system towards a leaflet of a host valvular structure.

Example 16. The system of any example herein, particularly any one of examples 13 to 15, wherein the snare loop is configured to transition to the expanded configuration when pushed distally out of the catheter lumen.

Example 17. The system of any example herein, particularly any one of examples 13 to 16, wherein the snare loop is made of a shape-memory material.

Example 18. The system of any example herein, particularly of example 17, wherein the shape-memory material comprises Nitinol.

Example 19. The system of any example herein, particularly any one of examples 13 to 18, wherein the snare loop is bendable relative to the snare shaft.

Example 20. The system of any example herein, particularly of example 19, wherein the snare loop is configured to extend at an angle of less than 180 degrees relative to the snare shaft, in the expanded configuration.

Example 21. The system of any example herein, particularly of example 19, wherein the snare loop is configured to extend at a 90-degrees angle relative to the snare shaft, in the expanded configuration.

Example 22. The system of any example herein, particularly any one of examples 13 to 21, wherein the snare loop is formed of a wire defining a snare loop wire thickness.

Example 23. The system of any example herein, particularly of example 22, wherein the catheter lumen defines a diameter that is not greater than a combination of twice the snare loop wire thickness and a thickness of the suture.

Example 24. The system of any example herein, particularly any one of examples 1 to 23, wherein the needle comprises a piercing tip configured to penetrate through a leaflet of a host valvular structure.

Example 25. The system of any example herein, particularly of example 24, wherein the host valvular structure is a valvular structure of a native heart valve.

Example 26. The system of any example herein, particularly of example 25, wherein the native heart valve is an aortic valve.

Example 27. The system of any example herein, particularly of example 24, wherein the host valvular structure is a valvular structure of a previously implanted prosthetic valve.

Example 28. A method comprising:
positioning a distal portion of a catheter inside a central opening of a host valvular structure;
exposing a snare loop out of the catheter by advancing a snare device comprising the snare loop through a catheter lumen of the catheter, thereby causing the snare loop to assume an expanded configuration thereof;
forming a puncture on a host leaflet of the host valvular structure using a needle comprising a needle lumen, thereby forming a puncture through the host leaflet;
distally advancing the needle through the puncture of the host leaflet and through an opening defined by the snare loop, to position a needle head of the needle distal to the snare loop;
advancing a grasper out of the needle lumen, wherein the grasper comprises a grasper head to which a free end portion of a suture is attached, and wherein the grasper is in a closed configuration when positioned in the needle lumen;
releasing the free end portion of the suture from the grasper head, such that the suture extends through the opening defined by the snare loop;
capturing the suture by the snare loop and pulling the suture into the catheter lumen, by proximally pulling the snare device to re-insert the snare loop into the catheter lumen; and
releasing a knot which is looped around the distal portion from the catheter by applying a distally oriented force by a push shaft disposed around the catheter.

Example 29. The method of any example herein, particularly of example 28, wherein the proximally pulling the snare device further comprises causing the snare loop to transition to a compacted configuration inside the catheter lumen.

Example 30. The method of any example herein, particularly of example 28 or 29, wherein the releasing the knot from the catheter further comprises forming a loop of the suture, the loop comprising a portion of the suture extending from the knot towards a free end of the host leaflet, bent around the free end and extending therefrom towards and through the puncture of the leaflet, and extending from the puncture towards the knot.

Example 31. The method of any example herein, particularly of example 30, further comprising, after the forming the loop, reducing the size of the loop, thereby compacting a part of the leaflet disposed inside the loop between the puncture and the free end.

Example 32. The method of any example herein, particularly of example 31, wherein the compacting comprises moving the free end of the host leaflet distally from a coronary ostium.

Example 33. The method of any example herein, particularly of example 31 or 32, wherein the releasing the knot from the catheter comprises disposing the knot directly around a portion of the suture that extends through the knot towards the catheter lumen.

Example 34. The method of any example herein, particularly of example 33, wherein the reducing the size of the loop comprises sliding at least one of the knot and the portion of the suture extending therethrough with respect to the other.

Example 35. The method of any example herein, particularly any one of examples 28 to 34, wherein the positioning the distal portion inside the central opening comprises maintaining the knot looped around the distal portion of the catheter.

Example 36. The method of any example herein, particularly any one of examples 28 to 35, wherein the forming the puncture through the host leaflet comprises maintaining the free end portion of the suture coupled to the grasper head.

Example 37. The method of any example herein, particularly of example 36, wherein the forming the puncture through the host leaflet comprises maintaining the grasper head inside the needle lumen.

Example 38. The method of any example herein, particularly any one of examples 28 to 37, further comprising, prior to the releasing the free end portion of the suture, maintaining a second portion of the suture extended from the knot towards the grasper head.

Example 39. The method of any example herein, particularly any one of examples 28 to 38, wherein the grasper head comprises a plurality of jaws pivotably movable relative to each other.

Example 40. The method of any example herein, particularly of example 39, wherein the plurality of jaws comprises two jaws connected by a hinge to each other.

Example 41. The method of any example herein, particularly of example 40, wherein the hinge comprises a pin.

Example 42. The method of any example herein, particularly of example 39 or 40, wherein the jaws are biased to an open configuration of the grasper head.

Example 43. The method of any example herein, particularly of example 42, wherein the releasing the free end portion of the suture comprises allowing the jaws to assume their biased state when the grasper head is positioned out of the needle lumen.

Example 44. The method of any example herein, particularly any one of examples 28 to 41, wherein the releasing the free end portion of the suture comprises actively extending the jaws away from each other.

Example 45. The method of any example herein, particularly any one of examples 28 to 44, further comprising, after the releasing the free end portion of the suture, retracting the needle out of the host leaflet.

Example 46. The method of any example herein, particularly any one of examples 28 to 45, further comprising, after the releasing the free end portion of the suture, proximally pulling the grasper away from the host leaflet.

Example 47. The method of any example herein, particularly of example 46, wherein the proximally pulling the grasper further comprises concealing the grasper head inside the needle lumen.

Example 48. The method of any example herein, particularly of example 47, wherein the concealing further comprises transitioning the grasper head to the closed configuration.

Example 49. The method of any example herein, particularly any one of examples 28 to 48, further comprising, prior to the releasing the knot, maintaining the knot proximal to a distal end of the catheter.

Example 50. The method of any example herein, particularly any one of examples 28 to 49, further comprising, prior to the releasing the knot, maintaining a distal edge of the push shaft proximal to the knot.

Example 51. The method of any example herein, particularly of example 50, wherein the releasing the knot comprises contacting the knot by the distal edge of the push shaft.

Example 52. The method of any example herein, particularly any one of examples 28 to 51, wherein the releasing the knot comprises distally sliding the knot over the distal portion.

Example 53. The method of any example herein, particularly any one of examples 28 to 52, wherein the releasing the knot comprises distally pushing the push shaft relative the knot.

Example 54. The method of any example herein, particularly any one of examples 28 to 53, wherein the releasing the knot comprises distally pushing the push shaft relative the catheter.

Example 55. The method of any example herein, particularly any one of examples 28 to 54, wherein the releasing the knot comprises proximally pulling the catheter relative to the push shaft.

Example 56. The method of any example herein, particularly any one of examples 28 to 55, wherein the causing the snare loop to assume the expanded configuration comprises extending the snare loop at an angle of less than 180 degrees relative to a snare shaft of the snare device.

Example 57. The method of any example herein, particularly any one of examples 28 to 55, wherein the causing the snare loop to assume the expanded configuration comprises extending the snare loop at an angle of 90 degrees relative to a snare shaft of the snare device.

Example 58. The method of any example herein, particularly any one of examples 28 to 57, wherein the proximally pulling the snare device comprises transitioning the snare loop to a compacted configuration thereof.

Example 59. The method of any example herein, particularly any one of examples 28 to 58, further comprising repeating the positioning the distal portion, the exposing the snare loop, the forming the puncture, the distally advancing the needle, the advancing the grasper, the releasing the free end portion, the capturing the suture, and the releasing the knot, with respect to another host leaflet of the host valvular structure.

Example 60. The method of any example herein, particularly any one of examples 31 to 34, wherein the reducing the size of the loop comprises locking the knot in place.

Example 61. The method of any example herein, particularly of example 60, further comprising, after the locking the knot, cutting the suture at a location proximal to the knot.

Example 62. The method of any example herein, particularly of example 61, further comprising, after the cutting, mounting a guest prosthetic valve within the host valvular structure, wherein the compacted part of the host leaflet allows blood to flow through a frame of the guest prosthetic valve to a coronary artery, which would otherwise be blocked by the host leaflet of the host valvular structure.

Example 63. The method of any example herein, particularly any one of examples 28 to 62, wherein the host valvular structure is a valvular structure of a native heart valve.

Example 64. The method of any example herein, particularly of example 63, wherein the native heart valve is an aortic valve.

Example 65. The method of any example herein, particularly any one of examples 28 to 62, wherein the host valvular structure is a valvular structure of a previously implanted prosthetic valve.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

We claim:

1. A leaflet folding system comprising:
a catheter defining a catheter lumen;
a snare device axially movable through the catheter lumen;
a needle defining a needle lumen;
a grasper axially movable through the needle lumen;
a suture comprising:
a knot looped around a distal portion of the catheter;
a first portion extending proximally from the knot; and
a second portion extending from the knot towards the grasper; and
a push shaft disposed around the catheter and axially movable relative to the catheter;
wherein the second portion comprises a free end portion and is releasably attached to the grasper at the free end portion; and
wherein the push shaft is configured to release the knot from the distal portion of the catheter by axially moving at least one of the push shaft and the catheter relative to the other, while the knot is engaged by the push shaft.

2. The system of claim 1, wherein the grasper comprises a grasper shaft and a grasper head at a distal end of the grasper shaft, and wherein the grasper head is configured to transition between a closed configuration and an open configuration.

3. The system of claim 2, wherein the grasper head is configured to release the free end portion of the suture in the open configuration.

4. The system of claim 2, wherein the grasper head comprises a plurality of jaws pivotably movable relative to each other.

5. The system of claim 1, wherein the snare device comprises a snare shaft and a snare loop at a distal end of the snare shaft, and wherein the snare loop is configured to transition between a compacted configuration and an expanded configuration.

6. The system of claim 5, wherein the snare loop is configured to transition to the expanded configuration when pushed distally out of the catheter lumen.

7. The system of claim 1, wherein the needle comprises a piercing tip configured to penetrate through a leaflet of a host valvular structure.

8. A method comprising:
positioning a distal portion of a catheter inside a central opening of a host valvular structure;
exposing a snare loop out of the catheter by advancing a snare device comprising the snare loop through a catheter lumen of the catheter, thereby causing the snare loop to assume an expanded configuration thereof;
forming a puncture on a host leaflet of the host valvular structure using a needle comprising a needle lumen, thereby forming a puncture through the host leaflet;
distally advancing the needle through the puncture of the host leaflet and through an opening defined by the snare loop, to position a needle head of the needle distal to the snare loop;
advancing a grasper out of the needle lumen, wherein the grasper comprises a grasper head to which a free end portion of a suture is attached, and wherein the grasper is in a closed configuration when positioned in the needle lumen;
releasing the free end portion of the suture from the grasper head, such that the suture extends through the opening defined by the snare loop;
capturing the suture by the snare loop and pulling the suture into the catheter lumen, by proximally pulling the snare device to re-insert the snare loop into the catheter lumen; and
releasing a knot which is looped around the distal portion from the catheter by applying a distally oriented force by a push shaft disposed around the catheter.

9. The method of claim 8, wherein the releasing the knot from the catheter further comprises forming a loop of the suture, the loop comprising a portion of the suture extending from the knot towards a free end of the host leaflet, bent around the free end and extending therefrom towards and through the puncture of the leaflet, and extending from the puncture towards the knot.

10. The method of claim 9, further comprising, after the forming the loop, reducing the size of the loop, thereby compacting a part of the leaflet disposed inside the loop between the puncture and the free end.

11. The method of claim 8, further comprising, after the releasing the free end portion of the suture, retracting the needle out of the host leaflet.

12. The method of claim 8, further comprising, after the releasing the free end portion of the suture, proximally pulling the grasper away from the host leaflet.

13. The method of claim 8, further comprising, prior to the releasing the knot, maintaining a distal edge of the push shaft proximal to the knot.

14. The method of claim 13, wherein the releasing the knot comprises contacting the knot by the distal edge of the push shaft.

15. The method of claim 8, wherein the releasing the knot comprises distally sliding the knot over the distal portion.

16. The method of claim 8, wherein the releasing the knot comprises distally pushing the push shaft relative the catheter.

17. The method of claim 8, wherein the releasing the knot comprises proximally pulling the catheter relative to the push shaft.

18. The method of claim 8, further comprising repeating the positioning the distal portion, the exposing the snare loop, the forming the puncture, the distally advancing the needle, the advancing the grasper, the releasing the free end portion, the capturing the suture, and the releasing the knot, with respect to another host leaflet of the host valvular structure.

19. The method of claim 10, wherein the reducing the size of the loop comprises locking the knot in place.

20. The method of claim 19, further comprising, after the locking the knot, cutting the suture at a location proximal to the knot.

21. The method of claim 20, further comprising, after the cutting, mounting a guest prosthetic valve within the host valvular structure, wherein the compacted part of the host leaflet allows blood to flow through a frame of the guest prosthetic valve to a coronary artery, which would otherwise be blocked by the host leaflet of the host valvular structure.
